(19) United States
(12) Patent Application Publication
(10) Pub. No.: US 2005/0065078 A1
(43) Pub. Date: Mar. 24, 2005

(54) Use of leptin for infant with low birth weight for prevention of obesity

(76) Inventors: Anthony Michael Cawthorne, Buckingham (GB); Joanne Claire Stocker, Buckingham (UA)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/488,129

(22) PCT Filed: Aug. 28, 2002
(86) PCT No.: PCT/GB02/03955

(30) Foreign Application Priority Data
Aug. 29, 2001 (GB) 0120884.2
May 23, 2002 (GB) 0211936.0

(51) Int. Cl. A61K 38/17

(52) U.S. Cl. 514/12

(57) ABSTRACT

The invention discloses the therapeutic and prophylactic administration of leptin to (i) an infant of low birth weight for age; (ii) a nursing mother of an infant, the infant having low birth weight for age; or (iii) a pregnant female predisposed to giving birth to an infant of low birth weight for age; for the prevention or treatment, in later life of the infant, of a metabolic disorder or other condition associated with low birth weight, such as type 2 diabetes, obesity, cardiovascular disease, gestational diabetes, impaired glucose tolerance, insulin resistance, hypertension or syndrome X. It is thought that the beneficial properties of leptin may be due at least in part to an effect on glucocorticoid metabolism, in particular to an increase in type 2 11β-hydroxysteroid dehydrogenase activity.

### FIELD OF THE INVENTION

The invention relates to the prevention of the development in later life of conditions, such as type 2 diabetes, obesity, cardiovascular disease, gestational diabetes, impaired glucose tolerance, insulin resistance, hypertension and syndrome X, associated with low birth weight.

### BACKGROUND OF THE INVENTION

Obesity, and especially upper body obesity, is a common and very serious public health problem in the United States and throughout the world. According to recent statistics, more than 25% of the United States population and 27% of the Canadian population are overweight (Kuczmarski, R. J., (1992) Amer. J. of Clin. Nutr. 55: 495S-502S; Reeder, B. A. et al, (1992) Can. Med. Assoc. J., 146: 2009-2019). Upper body obesity is the strongest risk factor known for people with type 2 diabetes, and is a strong risk factor for cardiovascular disease and cancer as well. Recent estimates for the medical cost of obesity are $150,000,000,000 world-wide. The prevalence of obesity in the population has become so serious that the Surgeon General has begun an initiative to combat the increasing adiposity in American society.

In addition, approximately 45% of males and 70% of females with NIDDM are obese, and their diabetes is substantially improved or even eliminated by weight reduction (see, e.g. Harris, (1991), Diabetes Care 14: 639-648). Both obesity and NIDDM are strongly heritable, though few of the predisposing genes have been identified. Hence the molecular genetic basis of these metabolically related disorders is an important, poorly understood problem.

Many obesity-induced pathologies can be attributed to the strong association with dyslipidemia, hypertension, and insulin resistance. Numerous studies have demonstrated that reduction in obesity by diet and exercise reduces these complications dramatically. Unfortunately, these treatments are largely unsuccessful with a failure rate nearing 95%.

Examination of the concordance rates of body weight and adiposity amongst mono- and dizygous twins or adoptees and their biological parents have suggested that the heritability of obesity (0.4-0.8) exceeds that of many other traits commonly thought to have a substantial genetic component, such as schizophrenia, alcoholism, and atherosclerosis (see e.g., Stunkard, et al. (1990), N. Engl. J. Med. 322: 1483-1487). Familial similarities in rates of energy expenditure have also been reported (see e.g., Bogardus, et al, (1986), Diabetes 35: 1.5). Genetic analysis in geographically delimited populations has suggested that a relatively small number of genes may account for the 30-50% of variance in body composition (see e.g. Moli, et al, (1991), Am. J. Hum. Genet. 49: 1243-1255.

The failure to control obesity may be due to the fact that the condition is strongly associated with genetically inherited factors that contribute to increased appetite, preference for highly caloric foods, reduced physical activity, and increased lipogenic metabolism. Thus, many people inheriting certain genetic traits are prone to becoming obese regardless of their efforts to combat the condition.

In addition, it has been shown that there is a strong association between low birth-weight and the later development of obesity, type 2 diabetes and hypertension. This association is particularly prevalent when the low birth weight is followed in adult life by the consumption of diets with a high fat content such as those typically found in Western countries and now increasingly found in developing countries that adopt Western dietary practices. This effect can be independent of any genetic effect since it has been shown that identical twins who are discordant for type 2 diabetes showed a lower body weight in those twins which were diabetic (Poulson et al, (1997), Diabetologia 40: 439-446). However, there is the potential that the low birth weight effect interacts with genetic components.

It has long been postulated that, when a mammal overeats, the resulting excess fat signals to the brain that the body is obese. These signals, in turn, cause the body to eat less and burn more fuel (Hervey, G. R., (1969), Nature 222: 629-631). This “feedback” model is supported by parabiotic experiments, which implicate a circulating hormone controlling adiposity.

In 1994, a new hormone, leptin, was described which is formed in fat cells and which is lacking in genetically overweight mice (ob/ob mice) (Zhang, Y., Proenca, R., Maffei, M., Barone, M., Leopold, L. and Friedman, J. M. (1994), Nature 372: 425-432). Human leptin and murine leptin are to a large extent identical. Injecting ob/ob mice with recombinantly prepared leptin leads to a reduction in nutrient intake and to a decrease in weight (Pelleymounter, M. A., Cullen, M. J., Baker, M. B., Hecht, R., Winters, D., Boone, T. and Collins, F. (1995), Science 269: 540-543). There has so far been no indication that mutations in the ob gene might be responsible for the frequent occurrence of obesity in humans. Systematic investigations have demonstrated that serum levels of leptin are increased in obese humans as they are in various animal models of obesity (Dagogo-Jack, S., Fanelli, C., Paramore, D., Brothers, J. and Landt, M. (1996), Diabetes 45: 695-698; Considine, R. V., Sinha, M. K., Heiman, M. L., Kriauciunas, A., Stephens, T. W., Nyce, M. R., Ohannesian, J. P., Marco, C. C., McKee, L. J., Bauer, T. L. and Caro, J. F. (1996), N. Engl. J. Med. 334: 292-295). For this reason, it is assumed that leptin is a feedback signal which informs the brain of the quantity of energy which is stored in the fat tissue. According to this assumption, it is then the function of the brain to decrease feed intake by inhibiting appetite, on the one hand, and to stimulate basal metabolism on the other. In human obesity, this regulatory circuit appears to be interrupted.

Leptin is primarily secreted from adipose tissue but is also secreted by the stomach (Bado et al, (1998), Nature 394: 790-793) and placenta (Hoggard et al, (1997), Proc.Nat. Acad. Sci. 94: 11075-11078). In addition to acting on receptors in the hypothalamus to regulate feeding and energy expenditure, it acts on a number of tissues including islet cells (Emilsson et al, (1997), Diabetes 46: 313-316), skeletal muscle (Lui et al, (1997), FEBS Lett. 411: 351-355), liver (Rossetti et al, (1997), J. Biol. Chem. 272: 27758-27763), adipose tissue (Chen et al, (2000), Biol. Neonate 78: 41-47), intestine (Morton et al, (1998), J. Biol. Chem. 273: 26194-26201).

Leptin also affects fertility (Chehab et al, (1996), Nature Genetics 12: 318-320). The sterility of male and female homozygous ob/ob mice was recognised since the original report of the ob mutation (Ingalls et al, (1950) J. Hered. 41: 317-318). ob/ob females are always sterile whereas ob/ob males can occasionally become fertile if maintained on a restricted diet (Lane et al, (1954), J. Heredity 45: 56-58). The ovaries of ob/ob females are capable of producing viable eggs when transplanted into lean female recipients (Hummel et al, (1957), Anat. Rec. 128: 569). Although early sexual development is normal, ovulation never follows and the mice remain prepubertal indefinitely. FSH, LH and testosterone levels are reduced in ob/ob females (Swerdloff et al, (1976), Endocrinology 98: 1359-1364), demonstrating the absence of a functional feedback from the hypothalamic-pituitary axis. Hypofunction of the pituitary gland in the female ob/ob mouse was demonstrated indirectly by showing that their in vivo uterine weights did not significantly change after bilateral ovariectomy (Runner et al, (1954), Genetics 39: 990-991); Drasher et al, (1955), J. Heredity 46: 209-212) but did, however, respond to exogenous estrogen. Pituitary extracts administered to ob/ob females induced ovulation and conception, but not implantation (Runner, M. N. (1954), Rec. Genet. Soc. Am. 23: 63-64) which was achieved following treatment with gonadotropic hormones (Runner et al, (1954), J. Heredity 45: 51-55). Furthermore, the administration of high doses of progesterone maintained pregnancy for 19 days p.c., but did not enable the mothers to deliver the fetuses except after administration of relaxin which stimulated parturition and lactation (Smithberg et al, (1956), J. Exp. Zool. 133: 441-458; Smithberg et al, (1957), J. Heredity 48: 97-100). The above findings demonstrated that the sterility of the ob/ob female is caused by an insufficiency of hormones at the hypothalamic-pituitary level rather than physical hindrance of copulatory activity by excess adipose tissue.

Kennedy and Mitra ((1963) J. Physiol. (London) 166: 408) proposed that puberty is linked to body weight and more specifically to fat storage which is as they conclude, one of the signals responsible for the initiation of hypothalamic control of ovarian function. Frisch and McArthur ((1974) Science 185: 949) related the loss or restoration of menstrual cycles in young girls to a minimum weight for height and reported that normal girls become relatively fatter from menarche to reproductive maturity. Therefore, these early and important findings established a relationship between initiation of reproduction and adiposity. In support of this relationship were the observations that very lean young female ballet dancers and college rowers (Frisch et al, (1980), NEJM 303: 17; Frisch et al, (1981), JAMA 246: 1559) have delayed puberty, whereas obese girls have an acceleration of puberty (Zacharias et al, (1970), Am. J. Obs. Gyn. 108: 833). Furthermore, the amenorrhea of extremely lean women was attributed to loss of fat and hypothalmic dysfunction (Vigersky et al, (1977), NEJM 297: 1141). Based on these findings, a “critical weight” hypothesis was suggested (Frisch et al, (1970), Science 109: 397) extending the assumption that a metabolic signal may be responsible for the initiation of reproduction. Moreover, adipose tissue has been viewed not only as an energy source but also as a direct regulator of female reproduction (R. E. Frisch, Adipose Tissue and Reproduction Progress in Reproductive Biology and Medicine, vol. 14 (1990)) since it converts androgens to estrogens via aromatisation (Sifteri, P. K. (1981), J. Endocrinology 89: 119).

Leptin has been shown to restore or enhance reproductive function in reproductively impaired male or female animals and accelerated the onset of puberty (Chelab, U.S. Pat. No. 5,773,416).

In humans and animals, plasma leptin increases early during gestation, derived primarily from the placenta (Masuzaki et al, (1997), Nature Medicine 3: 1029-1033). Although leptin and its receptor messenger RNA are expressed by the placenta (Hoggard et al, (1997), Proc. Natl. Acad. Sci. 94: 11075-11078) the role of increased leptin during pregnancy in maternal-fetal metabolism and intrauterine growth remains unclear. Schulz et al, (2000), BJOG 107: 1486-1491, concluded from studies in humans that circulating maternal leptin levels may provide a growth promoting signal for fetal development in late pregnancy and Yamashita et al, (2001), Endocrinology 142: 2888-2897, using an animal model of spontaneous gestation diabetes, showed that leptin administration during late gestation can prevent gestational diabetes but did not prevent fetal overgrowth.

Hales et al, (1991), Brit. Med. J. 303: 1019-1022, proposed the thrifty phenotype hypothesis to explain the epidemiological findings linking poor early (fetal and immediate post-natal) growth to an increased risk of loss of glucose tolerance in adults. These studies were extended to show that thinness at birth was linked to insulin resistance and the insulin resistance syndrome (Phillips et al, (1994), Diabetologia 37: 150-154). Moreover, it is clear that adult obesity strongly increases the risks posed by poor early growth.

Studies in an animal model of intra-uterine protein malnutrition (Ozanne and Hales, (1999), Proc. Natr. Soc. 58: 615-619) showed that these offspring were very prone to developing obesity on a high fat or palatable diet and this also increased blood pressure. These studies and others allowed the authors to conclude that intra-uterine protein malnutrition results in a metabolic programming of fetal tissues which is beneficial to survival under conditions of poor post-natal nutrition. However, if the organism moves into conditions of adequate or overnutrition then this will conflict with previous programming and conditions such as obesity, type 2 diabetes, hypertension and ischaemic heart disease may result. Low birth weight infants have a reduced pancreatic β-cell mass (Khan and Couper, (1994), Diabetes Care 17: 653-656). This reduction in β-cell mass cannot be restored fully by re-nutrition from weaning (Garofano et al, (1999), Diabetologia 42: 711-718). This represents a situation in which the offspring are predisposed to glucose intolerance and type 2 diabetes (Bertin et al, (1999), Am. J. Physiol. 277: E11-E17).

A factor that might mediate the in utero programming is increased fetal exposure to glucocorticoids (Bjorntorp et al (2000), Int. J. Obesity 24, Suppl. 2, S80-S85; Seckl et al (2000, Kidney Int. 57, 1412-1417).

High fetal glucocorticoid levels in the small baby syndrome may result from decreased expression of type 2 11β-hydroxysteroid dehydrogenase in the placenta. This enzyme normally protects fetal tissues from the high maternal levels of cortisol (corticosterone in rats) by catalysing the conversion of cortisol (corticosterone) to inert cortisone (11 dehydrocorticosterone) (Seckl et al (2000) Kidney Int. 57, 1412-1417). Type 1 11β-hydroxysteroid dehydrogenase catalyses the reverse reaction and results in the production of cortisol from cortisone. It has been shown that feeding dams on a low protein diet reduces placental type 2 11β-hydroxysteroid dehydrogenase activity (Langley et al (1996), Placenta 17, 169-172). In addition it has been shown that inhibition of this enzyme in pregnant mice by carbenoxolane results in small pups which become glucose intolerant in adult life (Lindsay et al (1996) Diabetologia 39, 1299-1305).

It has been shown that transgenic over-expression of 11βHSD-1 in adipose tissue of mice results in the development of visceral obesity, insulin resistant diabetes and hyperlipidaemia (Masuzaki et al (2001), Science 294, 2166-2170). Furthermore, 11βHSD-1 null mice have improved glucose tolerance (Morton et al (2001), J. Biol. Chem. 276, 41293-412300). These data indicate a role for 11βHSD-1 in the development of obesity, insulin resistance and hyperlipidaemia in adult animals and this is further supported by the finding that selective inhibitors of 11βHSD-1 such as BVT 2733 and BVT 14225 have anti-diabetic activity (Barf et al (2002), J. Med. Chem. Web release date 3 Aug. 2002, DOI 10,1021/JM025530f). However there are no indications in the literature that selective activation of 11βHSD-2 can be achieved.

Ravelli et al, (2000), Arch. Dis. Child 82: 248-252, studied the association between the method of infant feeding in the first weeks after birth and glucose tolerance, plasma lipid profile, blood pressure and body mass in adults aged 48-53 years, who were born in Amsterdam, Holland around the time of a severe period of famine. They found that exclusive breast feeding had a protective effect on glucose tolerance and on the high LDL/HDL ratio but body mass and systolic blood pressure was not affected. They suggest that growth factors or hormones present in breast milk might be responsible for the effect on lipid metabolism and note the fact that leptin is produced by the mammary gland and absorbed by the child (Casabiell et al, (1997), J. Clin. Endocrinol. Metab. 82: 4270-4273; Smith-Kirwin et al, (1998), J. Clin. Endocrinol. Metab. 83: 1810-1813) thereby potentially influencing growth and development. However, no direct effect of leptin was examined.

### SUMMARY OF THE INVENTION

We have now discovered that the nutritional programming caused by in utero growth retardation can be influenced by administration of leptin during pregnancy or post-parturition, increasing the offspring's resistance to the deleterious effects of a high fat diet in later life.

Accordingly the present invention provides the use of leptin, or a fragment or mimetic thereof, for the preparation of a medicament for administration to
(i) an infant of low birth weight for age;
(ii) a nursing mother of an infant, the infant having low birth weight for age;
or (iii) a pregnant female predisposed to giving birth to an infant of low birth weight for age;
for the prophylaxis, in later life of the infant, of a metabolic disorder or other condition associated with low birth weight.

Low birth weight is used herein to refer to low birth weight for age, as a result of in utero growth retardation, for example caused by inadequate nutrition. It is not intended to refer to low birth weight as a result of premature delivery. What is considered to be a normal birth weight for age will be determined by different factors in different species. For example, birth weight may vary according to e.g. sex and ethnicity of the offspring in humans. In general, birth weight may be considered to be low if it is within the lower two quintiles of the range observed in an appropriately matched population.

Where methods are referred to herein, the present invention further provides leptin, or a fragment or mimetic thereof, for use in those methods. Thus, inter alia, the present invention provides leptin for administration to (i) an infant of low birth weight for age;
(ii) a nursing mother of an infant, the infant having low birth weight for age;
or (iii) a pregnant female predisposed to giving birth to an infant of low birth weight for age;
for the prophylaxis, in later life of the infant, of a metabolic disorder or other condition associated with low birth weight.

Also provided is a method of prophylaxis, or medical treatment, comprising the administration of leptin to
(i) an infant of low birth weight for age;
(ii) a nursing mother of an infant, the infant having low birth weight for age;
or (iii) a pregnant female predisposed to giving birth to an infant of low birth weight for age;
for the prophylaxis, in later life of the infant, of a metabolic disorder or other condition associated with low birth weight.

The present invention further provides a method for preventing the development in later life of a metabolic disorder or other condition associated with low birth weight in an infant having low birth weight for age, the method comprising providing the infant with a prophylactically effective amount of leptin, or a fragment or mimetic thereof.

The present invention further provides a method for preventing the development in later life of an infant of a metabolic disorder or other condition associated with low birth weight, the method comprising providing a pregnant female, predisposed to giving birth to an infant having low birth weight for age, with a prophylactically effective amount of leptin, or fragment or mimetic thereof.

Conditions thought to be associated with low birth weight include type 2 diabetes, obesity, cardiovascular disease, gestational diabetes, impaired glucose tolerance, insulin resistance, hypertension and syndrome X, also known as insulin resistance syndrome.

Thus leptin, or a fragment or mimetic thereof, is provided to an infant, either in utero, or after birth, to prevent or ameliorate the development during later life of the metabolic disorder or other condition associated with low birth weight.

Leptin, fragments, and mimetic thereof, may be provided to an infant post partum, either directly, for example in admixture with milk, or indirectly, by administration to a nursing mother (i.e. a lactating female), in order that the active agent is delivered to the infant via the mother's milk. Clearly the female need not be the mother of the infant in question, as long as she is lactating and is capable of feeding the infant.

It is also believed that the administration of leptin, or a fragment or mimetic thereof, to a pregnant female can favourably influence the metabolic programming imposed upon her offspring. Thus leptin, or fragments or mimetics thereof may be provided to a pregnant female to prevent or ameliorate the development in her offspring of a metabolic disorder or other condition associated with low birth weight. In this regard, the leptin, or fragment or mimetic thereof, may be particularly effective when provided to the pregnant female during the third trimester of pregnancy.

A female predisposed to give birth to an infant of low weight for age is any female suspected to have a higher than normal chance of giving birth to such an infant. This assessment may be made based on her previous reproductive history, on physical assessment of the foetus during gestation, e.g. by means of a scan, or because of her nutritional status, lifestyle, or medical status. A number of factors affect the chance of females having small for date offspring. In humans, women who smoke or have asthma have a significantly greater chance than normal of having a small for date baby. Malnutrition, especially protein malnutrition, during pregnancy is also a significant cause of small for date offspring. In all species, females who have already had one or more small for date offspring will be at greater risk than normal of having further such offspring. Thus candidate females will be readily identifiable by clinicians, veterinarians, etc. Any suitable scanning technology may be applied during gestation to provide further data about whether or not offspring are likely to be small for date.

It is believed that the administration of leptin, or a fragment or mimetic thereof, to a pregnant female or nursing mother or directly to the infant during the pre-weaning period will favourably influence the metabolic programming of female offspring, so that when those female offspring themselves reproduce, gestational diabetes is prevented and normal offspring are produced.

The methods and compositions (e.g. medicaments and foodstuffs) provided by the present invention are suitable for application or administration to any mammal, but especially to humans and domestic animals, such as cats and dogs. Thus leptin, or a fragment or mimetic thereof, may be provided to infant humans or animals post partum to prevent or ameliorate the development during later life of metabolic disorders or other condition associated with low birth weight. Additionally, or alternatively, suitable active agents may be provided to a pregnant or lactating female.

The methods and compositions of the present invention are considered most efficacious when applied to infants in utero or before weaning. Thus in humans, infants will benefit most when treated in utero or in the first six months post partum, preferably in the first three months post partum. Suitable treatment times for other species may be calculated accordingly.

The present invention also provides a kit comprising leptin and instructions for administration to a pregnant female, a nursing mother, or an infant.

The invention further provides a method of preparing a medicament or foodstuff, comprising the step of admixing milk with leptin or a fragment or mimetic thereof.

Thus, for example, the present invention contemplates a method of adding leptin to milk derived from the same species, e.g. adding human leptin to human breast milk, e.g. full-term breast milk.

In alternative embodiments, the leptin is derived from a first mammalian species, and the milk from a different source, such as a second mammalian species. The leptin may be, for example, human leptin, or be from a domestic animal, such as canine or feline leptin. Thus in a preferred embodiment there is provided a method of preparing a medicament or foodstuff comprising the step of admixing human leptin and animal milk, preferably milk from an agricultural dairy animal, e.g. cow, sheep or goat milk.

The present invention further provides a medicament or foodstuff, comprising leptin, or a fragment or mimetic thereof, from a first mammalian species and milk from a different source, for example, from a second mammalian species, such as cows, goats, or sheep. Preferably the leptin is human, or from a domestic animal, e.g. feline or canine leptin. Thus in a preferred embodiment there is provided a medicament or foodstuff comprising human leptin and animal milk, e.g. cow, sheep or goat milk.

Agents capable of enhancing endogenous levels of leptin in the individual to which they are administered may also be used in any of the methods or compositions of the present invention. Such agents may increase the expression of endogenous leptin; for example, an diet which is deficient in essential fatty acids and high in saturated fatty acids and fatty acids having a low degree of polyunsaturation has been proposed to increase leptin levels (Korotkova, M, et al., (2002) Pediatric Research 52(1): 78-84). Thus the present invention further provides the use of an agent capable of enhancing endogenous levels of leptin for the manufacture of a medicament for administration to
(i) an infant of low birth weight for age;
(ii) a nursing mother of an infant, the infant having low birth weight for age;
or (iii) a pregnant female predisposed to giving birth to an infant of low birth weight for age;
for the prophylaxis, in later life of the infant, of a metabolic disorder or other condition associated with low birth weight. Treatment with such medicaments is intended to increase the levels of endogenous leptin in the infant, the nursing mother, or the pregnant female respectively. It will be clear to the skilled person that such agents may be used in all aspects of the invention, including administration in milk or other foodstuffs, etc.

Without wishing to be bound by any particular theory, it is thought that leptin may exert the effects described through an increase in the activity of placental type 2 11β-hydroxysteroid dehydrogenase (HSD-2). That is to say, leptin may increase the activity of HSD-2 which converts the stress hormone cortisol (or its equivalent, corticosterone, found e.g. in rodents) to the inactive molecule cortisone (or dehydrocorticosterone), and so may protect the developing foetus from the effects of placental cortisol/corticosterone. Throughout this specification the term “cortisol” is intended to embrace all equivalents of cortisol in other mammals, e.g. corticosterone.

Therefore in all aspects of the invention, leptin may be administered in conjunction with an agent capable of reducing cortisol bioactivity in the individual to which it is administered, either systemically, e.g. in the circulation, or in a specific tissue type. Such an agent may be a direct antagonist of cortisol which prevents the cortisol molecule from exerting its normal biological effects, e.g. by preventing interaction of cortisol with its receptor, or blocking the cortisol effector pathway. Such an agent may, for example, be a neutralising antibody capable of binding cortisol.

Alternatively the agent may be capable of modulating cortisol concentration, e.g. modulating synthesis or degradation of cortisol. For example the agent may increase the rate of inactivation of cortisol, e.g. by stimulating HSD-2 activity or expression. HSD-2 may be administered directly to increase the rate of inactivation of cortisol. Alternatively, the agent may decrease the rate of synthesis of cortisol by inhibiting HSD-1 activity or expression. Suitable inhibitors may include BT 2733 and BVT 14225 (Barf et al., 2002, above). Any suitable method for modulating expression of HSD-1 may be used.

In preferred embodiments, the agent is capable of inhibiting HSD-1 activity or expression. As the enzymes HSD-1 and HSD-2 have opposing activities, it is believed that an inhibitor of HSD-1 acting in combination with an activator of HSD-2 (such as leptin) will have a synergistic effect; that is to say the effect of using two such agents together will be greater than the sum of the effects obtained using each agent individually. The increased sensitivity of response may be similar to that observed with metabolic substrate cycles, in which a relatively small change in concentration of a regulator molecule capable of allosterically regulating both enzymes of a substrate cycle can cause a much larger change in the net flux through the pathway.

As it is thought that placental cortisol may promote the development of metabolic dysfunction later in the life of the infant, this aspect of the invention may be particularly suitable for administration to pregnant females, for in utero treatment.

### BRIEF DESCRIPTION OF THE FIGURES

FIG. 1 shows growth of offspring of female rats fed on an 8% protein diet, and given a saline infusion from day 14 of pregnancy.

FIG. 2 shows growth of offspring of female rats fed on an 8% protein diet, and given an infusion of leptin from day 14 of pregnancy.

FIG. 3 shows plasma leptin concentration in female rats fed on normal 20% protein diet (NPS) and on an isocaloric low 8% protein diet with either an infusion of saline (LPS) or leptin (LPL) from day 14 of pregnancy and throughout lactation.

FIG. 4 shows the placental weights in female rats fed on a normal 20% protein diet (NPS) or on an isocaloric low 8% protein diet with either an infusion of saline (LPS) or leptin (LPL) from day 14 of pregnancy.

FIG. 5 shows the birthweight of offspring from mothers fed on a normal 20% protein diet (NPS) or fed on an isocaloric low 8% protein diet with either an infusion of saline (LPS) or leptin (LPL) from day 14 of pregnancy.

FIG. 6 shows the body weight of pups during lactation from mothers fed on a normal 20% protein diet (NPS) and on an isocaloric 8% protein diet with either an infusion of saline (LPS) or leptin (LPL) from day 14 of pregnancy and throughout lactation.

FIG. 7 shows growth of the male offspring fed on a high fat diet, from mothers that had been fed on a normal 20% protein diet.

FIG. 8 shows growth of the male offspring fed on a high fat diet, from mothers that had been fed on an isocaloric low (8%) protein diet, and given a saline infusion from day 14 of pregnancy and throughout lactation.

FIG. 9 shows the growth of the male offspring fed on a high fat diet, from mothers that had been fed on an isocaloric low (8%) protein diet, and given an infusion of leptin from day 14 of pregnancy and throughout lactation.

FIG. 10 shows the fasting insulin concentration of male offspring fed on a high fat diet, from mothers that had been fed on a normal 20% protein diet.

FIG. 11 shows fasting insulin concentration of male offspring fed on a high fat diet, from mothers that had been fed on an isocaloric low (8%) protein diet and given a saline infusion from day 14 of pregnancy and throughout lactation.

FIG. 12 shows the fasting insulin concentration of male offspring fed on a high fat diet, from mothers that had been fed on an isocaloric low (8%) protein diet and given an infusion of leptin from day 14 or pregnancy and throughout lactation.

FIG. 13 shows the integrated blood glucose concentration during a glucose tolerance test in 6 and 12 month old rats.

FIG. 14 shows the integrated plasma insulin concentration during a glucose tolerance test in 6 and 12 month old rats.

FIG. 15 shows the plasma corticosterone levels in dams during pregnancy and lactation.

FIG. 16 shows the activity of 11β-hydroxysteroid dehydrogenase-1 (HSD-1) and 11β-hydroxysteroid dehydrogenase-2 (HSD-2) in the placenta at day 20.5 of pregnancy.

### DETAILED DESCRIPTION OF THE INVENTION

Conditions which may be prevented or ameliorated in later life by the methods and compositions of the present invention include any metabolic disorder associated with low birth weight for date, including type 2 diabetes, obesity, cardiovascular disease, gestational diabetes, impaired glucose tolerance, insulin resistance, hypertension and syndrome X.

Syndrome X, also known as insulin resistance syndrome, is a description given to a variety of symptoms of metabolic dysregulation which precede development of a major metabolic disorder such as type II diabetes. Syndrome X is characterised by one or more of glucose intolerance, increased plasma triglycerides, increased low and very low density lipoproteins, decreased high density hdl-cholesterol, post-prandial lipaemia, increased serum uric acid, slight increase in blood pressure, and increased plasminogen activator inhibitor 1.

Active agents suitable for use in the present invention include full-length leptin polypeptides including modified molecules containing adducts such as dextran, fatty acids or pegylated moieties, or biologically active fragments or mimetics thereof. Leptin polypeptides may be isolated from physiological sources, or recombinantly produced. Recombinant polypeptides may be produced in any appropriate expression system, such as mammalian, insect, bacterial or yeast expression systems, and may be produced with or without their naturally occurring secretion signal peptides.

Methods for cloning and purifying leptin compounds suitable for use in the present invention have been described in the scientific literature. See, for example, Pelleymounter et al, (1995), Science 269: 540-543; Halaas et al, (1995), Science 269: 543-546; Campfield et al, (1995), Science 269: 546-549 and Chehab et al, (1996), Nat. Gen. 12: 318-320.

The present invention is applicable for the treatment of mammals, and in preferred embodiments for the treatment of humans and domestic animals, such as dogs and cats. When natural or recombinant leptin, or fragments thereof are used, they will preferably be derived from the species which it is to be used to treat. Thus humans will preferably be treated with human leptin. However, leptins may be used to treat species other than those from which they are derived. In preferred embodiments, the leptin is derived from human, cat, dog, mouse, pig, anthropoid ape, bovine or ovine sources. However, clearly other forms of leptin can be used for treatment of other species.

Biologically active leptin fragments and mimetics are also suitable for use in the present invention. Fragments include peptides derived from full-length leptin polypeptides, and truncated recombinant forms of leptin. Suitable leptin fragments include those disclosed in U.S. Pat. No. 6,187,751, WO97/46585 and WO00/11173.

Mimetics are considered to be active agents sharing one or more biological activities with leptin. They may be naturally occurring polypeptides sharing biological activities with leptin, such as for example, human obesity protein homologue-1, as disclosed in WO01/25428, and ciliary neurotrophic factor activators including Axokine as disclosed in WO98/22128.

Alternatively, a mimetic may be a recombinant leptin polypeptide having amino acid substitutions, deletions or insertions relative to a native leptin sequence, which substantially retain or have enhanced leptin biological activity.

Alternatively a mimetic may be a synthetic small molecule, peptide or polypeptide drug capable of exerting one or more biological effects of leptin. The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, eg peptides may be unsuitable active agents for oral compositions as they tend to be quickly degraded by proteases in the alimentary canal. Mimetic design, synthesis and testing is generally used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, eg by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in vivo or clinical testing.

Leptins, leptin fragments, or mimetics fused to non-leptin polypeptides, or conjugated to small-molecule carriers are also suitable for use in the present the invention. The non-leptin polypeptide or carrier may influence biological availability or pharmacokinetics, such as half life in the blood stream. Suitable fusion partners or carriers will be well known to the person skilled in the art, and include for example immunoglobulin Fc regions.

Without wishing to be bound by any particular theory, it is believed that administration of leptin to offspring following parturition is effective in overcoming the metabolic programming imposed on the offspring in utero. Thus, in one aspect, the leptin, or fragment or mimetic thereof, is provided to an infant to prevent or ameliorate the development during later life of a metabolic disorder or other condition associated with low birth weight. In this aspect, the leptin, fragment or mimetic thereof, may be provided to an infant in milk.

Additionally or alternatively, the leptin, fragment or mimetic thereof, may be provided to an infant indirectly, by administration of the agent to a nursing mother, in order that the active agent is delivered to the infant in the mother's milk.

It is also believed that the administration of leptin to a pregnant female can favourably influence the metabolic programming imposed upon her offspring. Thus in a further aspect of the present invention the leptin, or fragment or mimetic thereof is provided to a pregnant female to prevent or ameliorate the development in her offspring of the metabolic disorder or other condition associated with low birth weight. In this regard, the leptin, or fragment or mimetic thereof, may be particularly effective when provided to the pregnant female during the third trimester of pregnancy.

Leptin may be administered orally to an infant, either formulated as a pharmaceutical, or in admixture with milk. This is because proteins are absorbed intact from the infant alimentary canal into the bloodstream, without proteolysis or degradation. Protein given orally to an adult will typically be degraded in the alimentary canal. Therefore when an active agent to be administered to a pregnant or nursing female is a protein, peptide or polypeptide, it will typically be formulated as a pharmaceutical for administration by other than oral means.

Pharmaceutical compositions may comprise, in addition to one or more active agents, a pharmaceutically acceptable excipient, carrier, buffer, stabiliser or other materials well known to those skilled in the art. Such materials should be non-toxic and should not interfere with the efficacy of the active ingredient.

Suitable pharmaceutically acceptable carriers are as dictated by conventional practice such as those disclosed in GB 2292382 or in International Patent Application Publication number WO 94/01420. They also include pharmaceutically acceptable carriers, which are compatible with incorporation into milk powders and liquid milk. This is particularly appropriate for administration direct to an infant. Leptin, and fragments and mimetics thereof may be provided in admixture with animal milk or human breast milk, or in any suitable formulation of powdered or dried milk, either prior to or after reconstitution.

As set out above, the invention further provides a method of preparing a medicament or foodstuff, comprising the step of admixing milk with leptin or a fragment or mimetic thereof.

Thus, for example, the present invention contemplates a method of adding leptin to milk derived from the same species, e.g. adding human leptin to human breast milk, e.g. full-term breast milk.

In alternative embodiments, the leptin is derived from a first mammalian species, and the milk from a different source, for example from a second mammalian species. The leptin may be, for example, human leptin, or be from a domestic animal, such as canine or feline leptin. Thus in a preferred embodiment there is provided a method of preparing a medicament or foodstuff comprising the step of admixing human leptin and animal milk, e.g. cow, sheep or goat milk.

However, the term ‘milk’ is used herein to refer to milk from any mammalian species, in treated or untreated form, as well as to milk substitutes intended to provide nutrition for infants. Thus the term ‘milk’ encompasses human or animal milk in full, semi-skimmed or skimmed form, in liquid, powder or concentrate form, pasteurised or unpasteurised. Also encompassed by the term ‘milk’ is infant formula, which typically comprises milk derivatives with additional nutritional supplements, which are readily commercially available, as well as artificial milk substitutes such as soya milk, which typically comprise non-animal protein, optionally supplemented by sugars, other carbohydrates, fats, and other nutritional additives.

Typically the mode of admixture will depend upon the form of the milk to which the leptin is to be added, and will be chosen so as to retain leptin activity in the final mixture. For example, milk is often sterilised by heat treatment, e.g. by pasteurisation. Leptin activity may be affected by heat treatment, so leptin may be added to milk after sterilisation, in order that leptin activity is not adversely affected by the sterilisation process.

When administered other than in milk, e.g. to a pregnant or nursing female, the precise nature of the carrier or other material may depend on the route of administration. The active agent may be administered subcutaneously, intradermally, intravenously, intramuscularly, intraperitoneally, via pulmonary delivery, via intranasal delivery, transdermally, orally, via controlled release, via pump or by any conventional route of administration for polypeptide drugs. Typically the agent will be administered continuously during the period of administration, i.e. being delivered at least once per day or via controlled release techniques via a transdermal patch or a sustained release injectable formulation providing, for example, a 28 d supply.

Therapeutic compositions may comprise a solution of leptin, or a fragment or mimetic thereof, dissolved or suspended in an acceptable carrier, preferably an aqueous carrier. A variety of aqueous carriers may be used, e.g., water, buffered water, 0.8% saline, 0.3% glycine, hyaluronic acid and the like. These compositions may be sterilised by conventional, well known sterilisation techniques, or may be sterile filtered. The resulting aqueous solutions may be packaged for use as is, or lyophilised, the lyophilised preparation being combined with a sterile solution prior to administration. The compositions may contain pharmaceutically acceptable auxiliary substances as required to approximate physiological conditions, such as pH adjusting and buffering agents, toxicity adjusting agents, wetting agents and the like, for example, sodium acetate, sodium lactate, sodium chloride, potassium chloride, calcium chloride, sorbitan monolaurate, triethanolamine oleate, etc.

The concentration of the leptin, fragment, or mimetic thereof in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

When administered in milk, the leptin, or fragment or mimetic thereof will typically have an activity corresponding to a solution in milk of up to 30 ng/ml of full-length leptin from the same species as the subject. Preferably, especially when intended for administration to human infants, the leptin activity will correspond to that of a solution in milk of 2 to 20 ng/ml of full-length human leptin, preferably 10 to 20 ng/ml.

For solid compositions, conventional non-toxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable non-toxic composition is formed by incorporating any of the normally employed excipients, such as those carriers previously listed, and generally 10%-95% of active ingredient, that is, one or more leptin compounds of the invention, and more preferably at a concentration of 25%-75%.

For aerosol administration, the active agent is preferably supplied in finely divided form along with a surfactant and propellant. Typical percentages of leptin are 0.01%-20% by weight, preferably 1%-10%. The surfactant must, of course, be non-toxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhdric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

The therapeutic compositions of the invention can additionally be delivered in a controlled release system such as a depot-type system, an encapsulated form, or an implant by techniques well-known in the art. The compositions of the invention can also be delivered via a pump, such as a minipump, to pregnant female host and/or to preweaned offspring.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

For example, full length leptin will usually be administered in a dosage from 0.1 ng/kg body weight to 100 mg/kg body weight to either or both the pregnant female or the offspring. Amounts effective for use will depend on, for example, the particular active agent, the method of formulation, the manner of administration, and the weight of the patient.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in target cells by expression from an encoding gene introduced into the cells, eg in a viral vector (a variant of the VDEPT technique—see below). The vector could be targeted to the specific cells to be treated, or it could contain regulatory elements which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, eg an enzyme, in a vector by expression from encoding DNA in a viral vector (see for example, EP-A-415731 and WO 90/07936).

When the methods of the present invention are to be used to administer active agents to a pregnant female, the female will usually suffer from a nutritional or other disorder or a lifestyle disorder such as smoking that results in the production of a small for date offspring. The female host may be lean, of normal adiposity or obese. In a preferred embodiment, the active agent is administered to the pregnant host during the 3^{rd} trimester.

When the methods of the present invention are used to administer active agents to offspring post-partum, either via mother's milk or via direct administration, the active agent will typically be administered to the offspring during at least a portion of the first 3 months of life. Preferably, the active agent is administered throughout the first three months of life.

Thus when the active agent is to be administered via mother's milk, the active agent will typically be administered to a female host during at least a portion of the first 3 months of lactation, and preferably throughout the entire period of lactation.

The present invention also provides a method of preventing the development of a metabolic disorder or other condition associated with low birth weight in a patient, the method comprising providing the patient with a prophylactically effective amount of leptin, or a fragment or mimetic thereof.

Also provided is a method of preventing the development of a metabolic disorder or other condition associated with low birth weight in an infant, the method comprising providing a pregnant female with a prophylactically effective amount of leptin, or fragment or mimetic thereof.

In all aspects of the invention, agents capable of reducing cortisol (or corticosterone) bioactivity may be administered in conjunction with the leptin, fragment or mimetic thereof. Thus such agents may be administered as part of a medicament or foodstuff, or in milk. The agent capable of reducing cortisol bioactivity and leptin may be administered individually or together, in the same or different foodstuffs, milk formulations or medicaments. Preferably the agent capable of reducing cortisol bioactivity is administered in a pharmaceutically acceptable quantity and form.

### EAXMPLE 1

Pregnant rats were fed on either a control diet containing 20% protein or an isocalorific diet containing 8% protein throughout pregnancy and lactation. On day 14 of pregnancy (at the beginning of third trimester) rats on the 8% protein diet were assigned to a control group or a leptin-treated group. Each rat received a continuous infusion of saline or leptin for 28 days via a subcutaneous minipump. The group receiving leptin were given 1 mg/kg/day as a continuous infusion from an Alzet mini-pump model 2ML4, at 2.5 microlitres per hour for 28 days

Pups were weaned at day 21 onto the control 20% protein diet and then at 6 weeks of age transferred to either a control or high fat diet until 8 months of age.

Leptin infusion significantly elevated plasma levels in the pregnant rats fed on a low protein diet. Leptin infusion reduced voluntary food intake in the pregnant rats fed on a low protein diet during pregnancy and reduced the post-birth maternal body weight.

Leptin treatment did not affect the live litter size, the birth weight of pups or the placental weight.

However, the birth weight of pups from mothers receiving the 8% protein diet was significantly lower than the birth weight of pups on a 20% protein diet. Similarly the weight at 21 days of age of pups from mothers on the 8% protein diet was less than those from mothers on the 20% protein diet but leptin treatment had no additional influence.

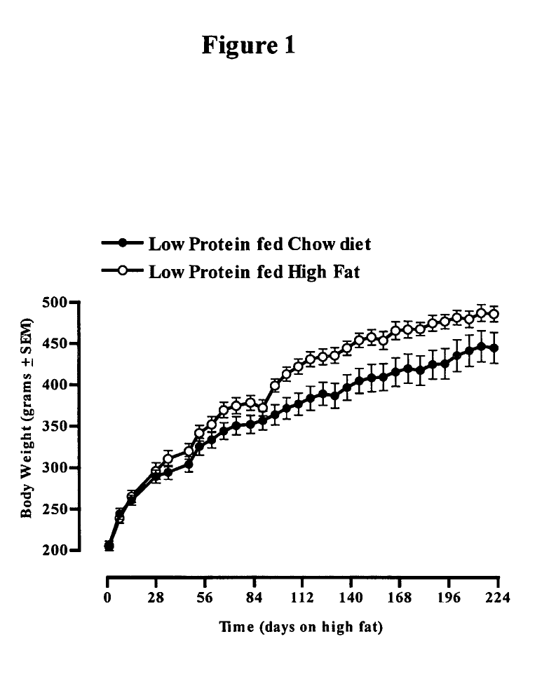

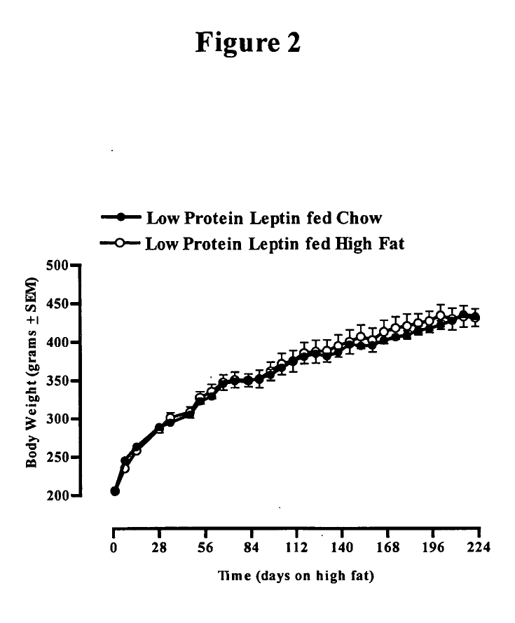

In offspring from mothers fed on an 8% protein diet who received the saline infusion during the 3^{rd} trimester and during lactation the consumption of a high fat diet from 6 weeks of age onwards induced obesity relative to similar animals given a normal chow diet (FIG. 1). In contrast, the offspring from mothers given the same 8% protein diet but who received an infusion of leptin were completely resistant to the obesity inducing effects of a high fat diet (FIG. 2).

### EXAMPLE 2

Pregnant Wistar rats were fed on either a normal (20% protein) diet or an isocaloric diet containing 8% protein throughout pregnancy and lactation. From day 14 of pregnancy they received saline or leptin (2 mg/kg/day) via a subcutaneous minipump (Alzet Corp.) for 28 days. All pups were weaned at 21 days old onto a 20% protein diet and at 6 weeks of age some of the pups were transferred to a high fat diet (60% of calories provided by fat).

A glucose tolerance test was performed at 6 months of age. In addition measurements were made of glucose and insulin throughout the study and during the glucose tolerance test to assess insulin sensitivity.

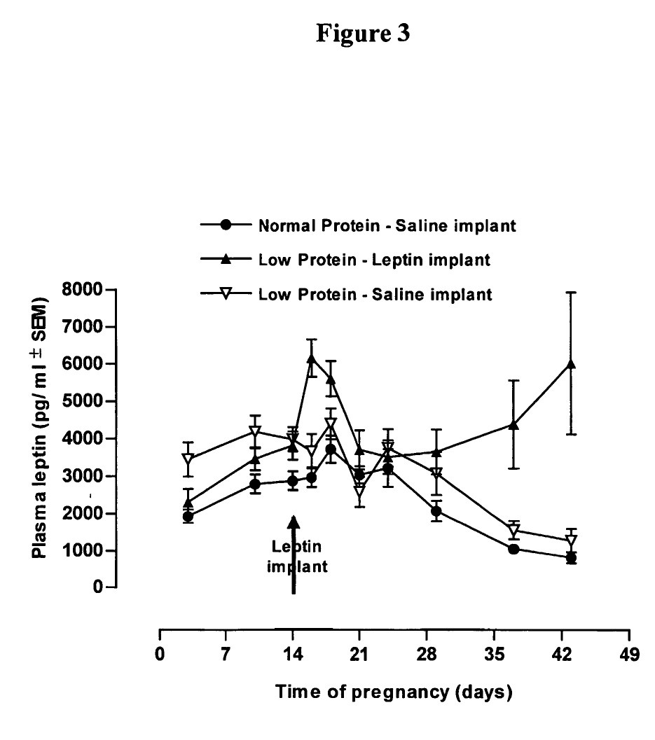

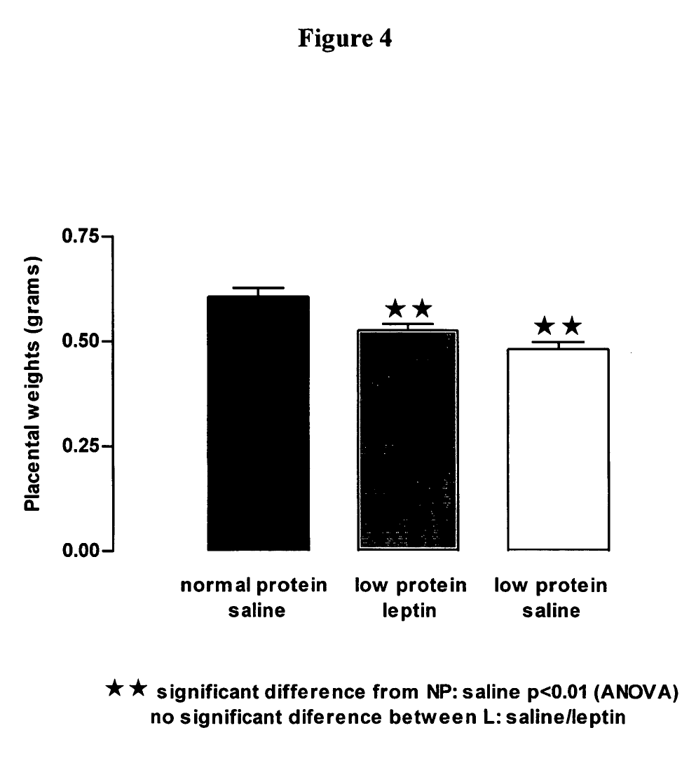

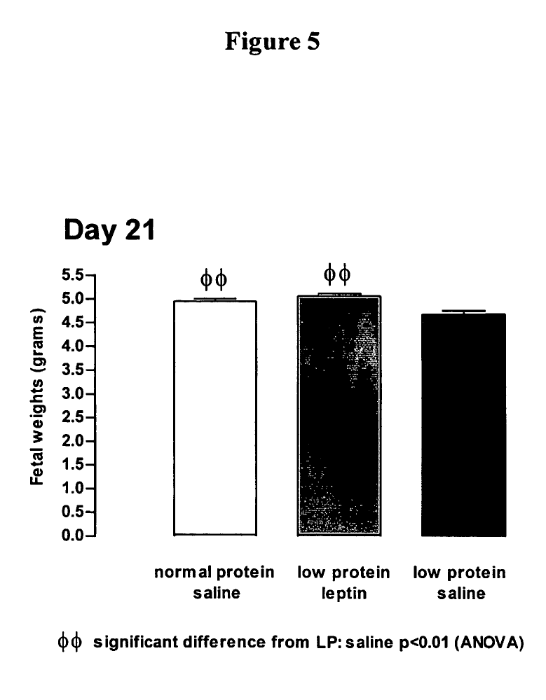

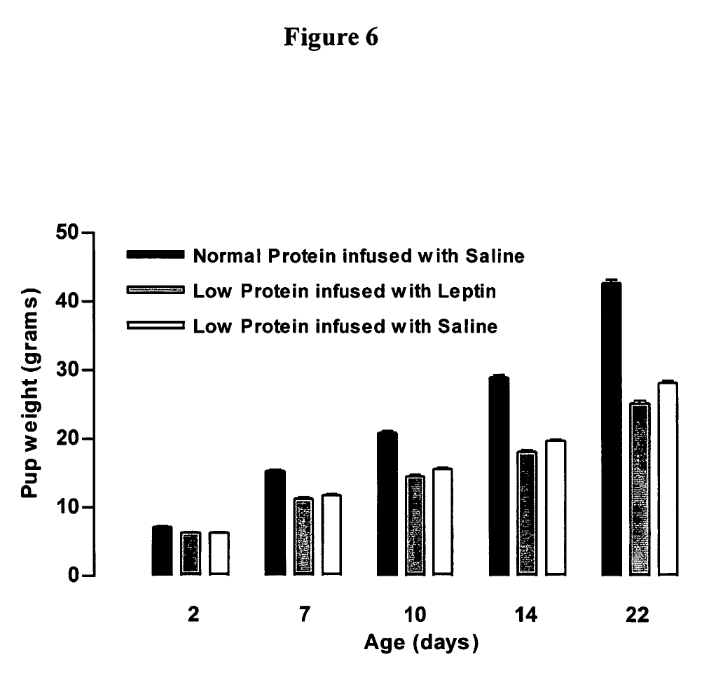

Plasma leptin concentrations in the mothers given leptin were increased 5-fold (FIG. 3) relative to the concentration in mothers not given leptin, but had only a minor effect on maternal food intake and body weight. The litter size was the same in all three groups. Placental weights were lower in low protein diet mothers (FIG. 4) but birth weight of offspring of low protein fed rats given leptin was the same as offspring from rats given a normal protein diet and greater than those from rats given a low protein diet and infused with saline (FIG. 5). However, post-birth during lactation the weight of pups from leptin-treated mothers given a low protein diet was the same as that of pups from mothers on the same diet but not given leptin (FIG. 6). At 2 days of age pancreatic insulin content in pups from saline-treated mothers given a low protein diet were significantly lower (4.0±0.7 μg/pancreas) than in pups from mothers on the normal protein diet (6.2±1.4 μg/ pancreas). Leptin treatment of the low protein diet mothers resulted in a significant increase in the pancreatic insulin content in 2 day old pups (6.4±1.0 μg/pancreas). Glucagon and amylin content of the pancreas of 2 day old pups was not affected by prior dietary manipulation or leptin administration to the mother. Glucose tolerance was undertaken at 6 weeks of age. The low protein saline offspring were more insulin sensitive than the normal protein saline offspring and this was not altered by leptin treatment

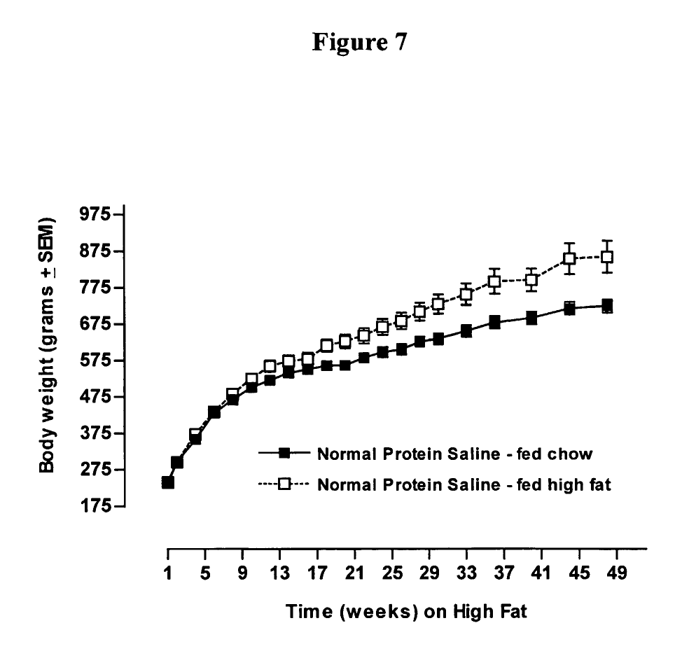

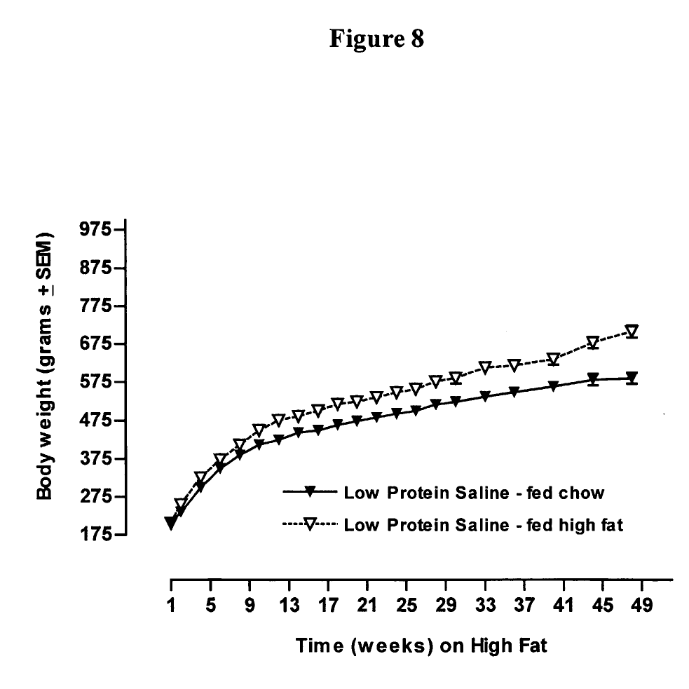

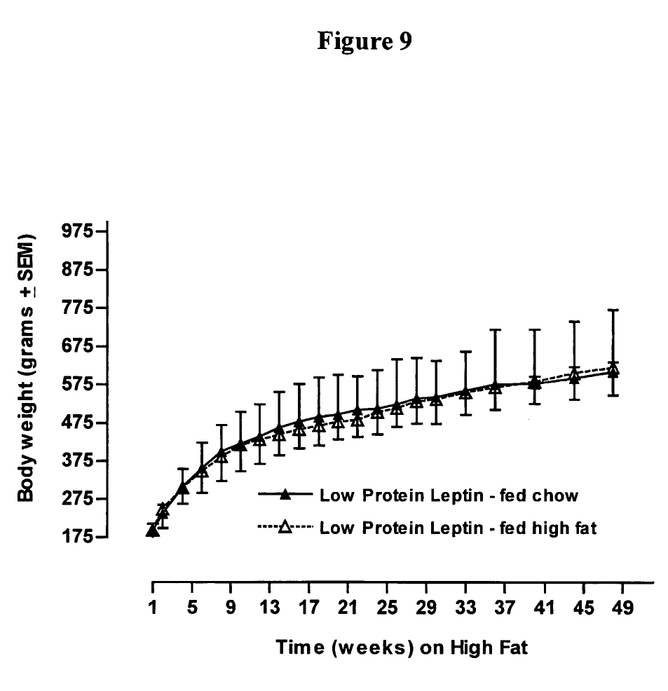

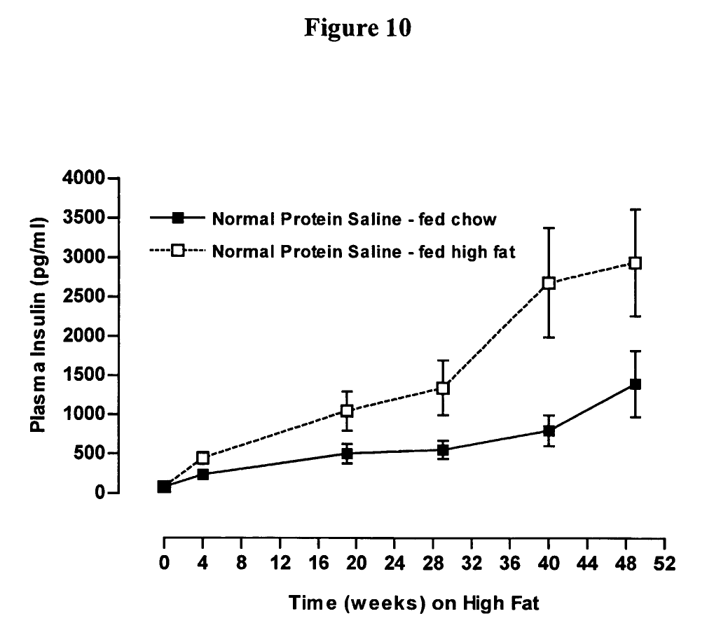

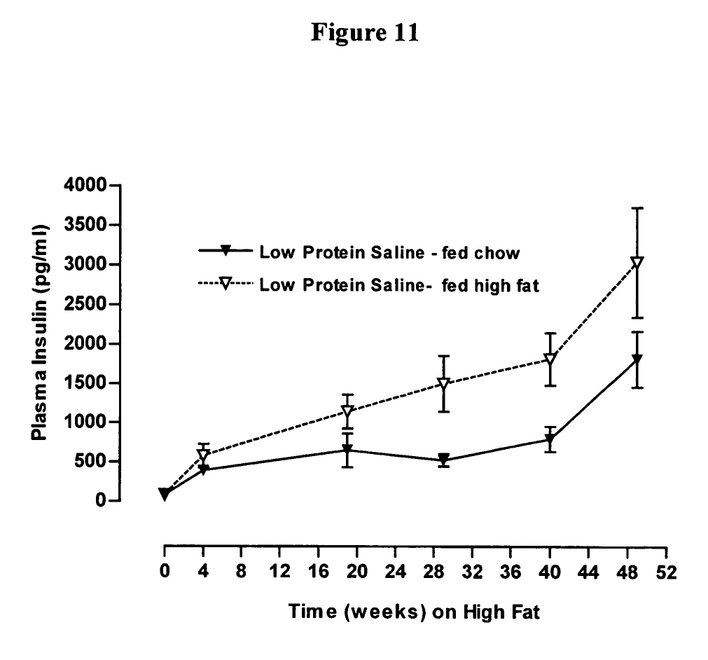

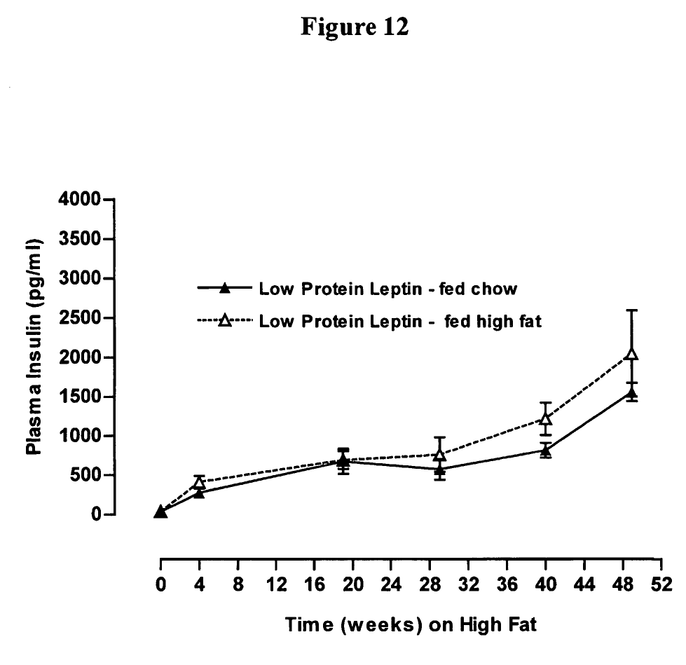

At 6 weeks of age, some of the rats were placed on a high fat diet whereas the remainder continued on the normal protein diet. The high fat diet mirrors in composition the typical Western diet. The high fat diet induced obesity in offspring from mothers given both the normal protein (FIG. 7) and low protein diet (FIG. 8), but the additional treatment of the low protein diet mothers with leptin prevented the high fat diet induced obesity (FIG. 9). In addition the high fat diet induced fasting hyperinsulinaemia in offspring from mothers given either the normal protein (FIG. 10) or low protein diet (FIG. 11), but leptin treatment of the mother prevented this in the low protein offspring (FIG. 12). Since fasting glucose concentrations were not different between groups, it is clear that the high fat diet induced insulin resistance but leptin treatment of the mothers resulted in rats that resisted high fat diet-induced insulin resistance.

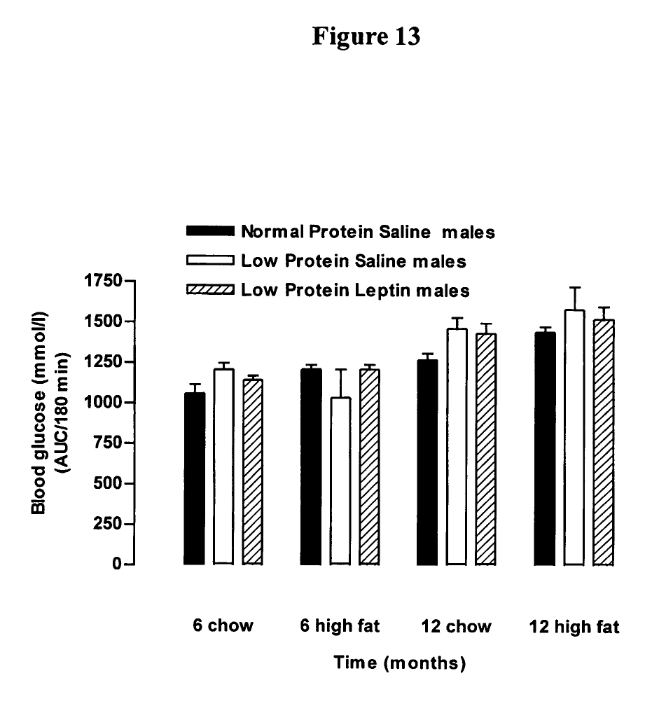

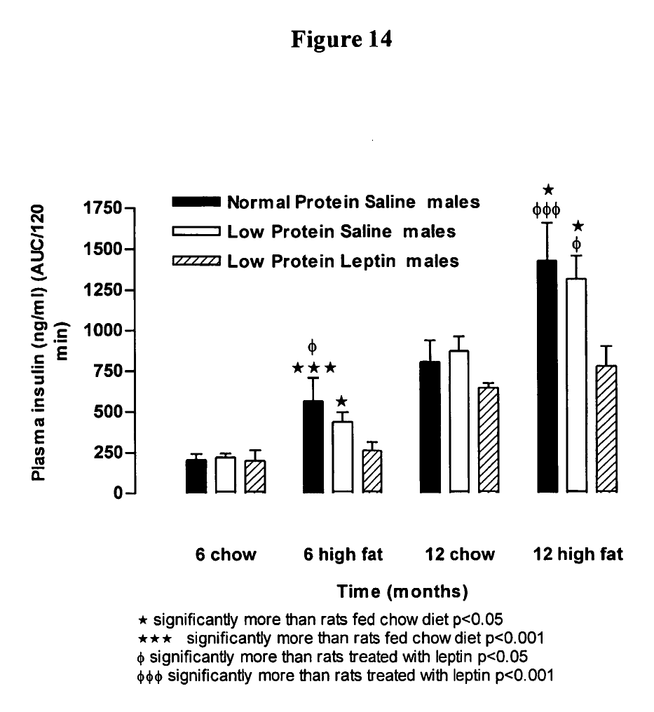

At 6 months and 12 months of age, the rats were given an intraperitoneal glucose load to determine glucose tolerance. There was no difference in the glucose tolerance curves for the 6 groups (FIG. 13) but insulin concentration in the high fat diet fed rats from mothers on both a normal protein and a low-protein diet (given saline) were markedly elevated (939±221 pmol/l and 872±139 pmol/l respectively) at 30 mins after glucose administration. Leptin-treatment of the low protein diet mothers in the third trimester and during lactation prevented this glucose-induced hyperinsulinaemia (464±77 pmol/l) again demonstrating a prevention of high fat diet induced insulin resistance. The integrated areas of the plasma insulin concentration during the period 0-2 h post the glucose load are given in FIG. 14. The high fat diet fed offspring of the leptin-treated dams given the low protein diet had a significantly reduced insulin output relative to high-fat diet fed offspring of dams given saline plus the low-protein diet or the normal protein diet. This indicates increased insulin sensitivity in the offspring of leptin-treated dams.

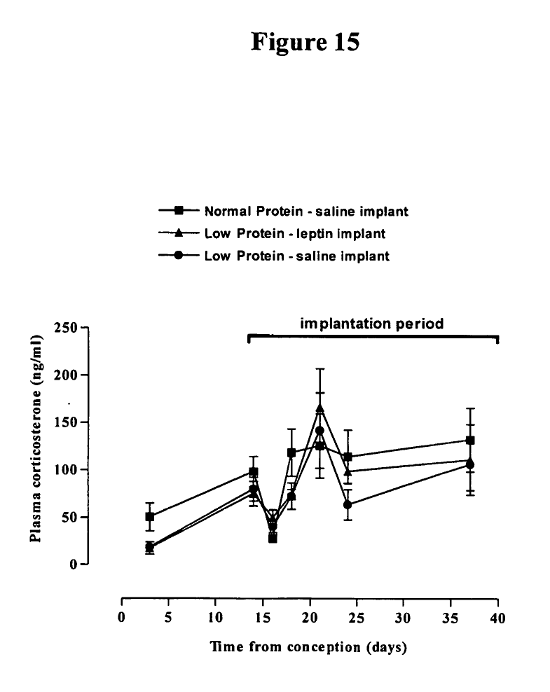

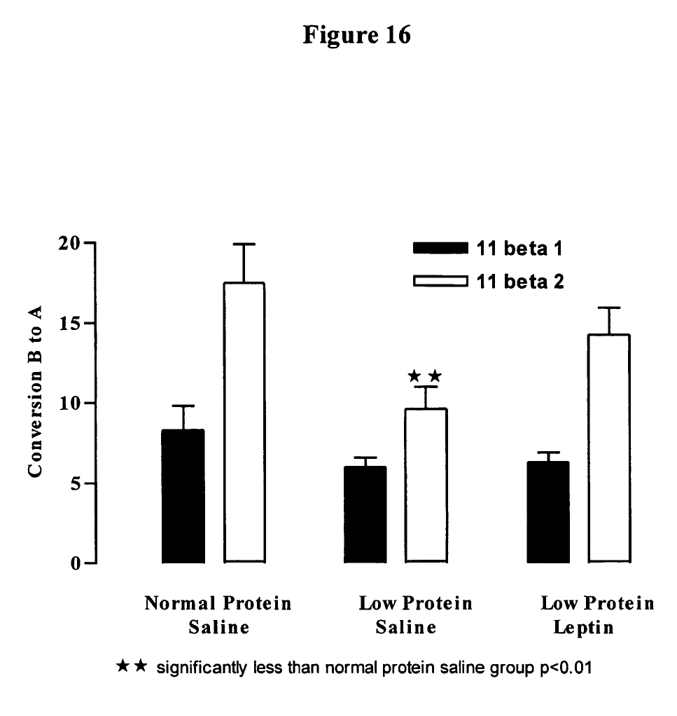

The corticosterone levels of the dams given either the normal protein diet or the low protein diet did not differ irrespective of the administration of leptin (FIG. 15). Additionally the activity of placental 11β-hydroxysteroid dehydrogenase-1, which catalyses the formation of corticosterone from the inactive dehydrocorticosterone did not differ between the groups. However, the activity of 11β-hydroxysteroid dehydrogenase-2 , which catalyses the conversion of corticosterone to inactive dehydrocorticosterone was reduced in the placenta of rat fed on the low protein diet and given saline relative to that of placentas from normal protein-fed dams. The administration of leptin to the low protein animals prevented the significant reduction in 11β-hydroxysteroid dehydrogenase-2 enzyme activity (FIG. 16).

Methods

Animals

All animal procedures were conducted under the British Home Office Animals (Scientific Procedures) Act. Pregnant Wistar rats (Charles River, UK Ltd, Margate, UK) (initial weight 200-225 g) were received time-mated at day 1 of gestation (taken when vaginal plugs were detected), housed individually and maintained at 22° C. on a 12:12 h light:dark cycle. The rats were fed either a diet containing 20% (w/w) protein or an isocaloric diet containing 8% (w/w) protein (Hope Farms, Woerden, Netherlands) The composition and source of the diets were as described previously (Snoeck, A. et al., Biol Neonate 57, 107-18 (1990)) throughout pregnancy and lactation. The deficit in energy of the low protein diet was made up by an increase in its carbohydrate content. From day 14 of pregnancy normal protein-fed rats received saline and low protein-fed rats either saline or leptin (2 mg/kg/d in physiological saline; PeproTech EC Ltd, London, UK) via a subcutaneously implanted Alzet™ minipump (Charles River, UK Ltd, Margate, UK) for 28 days.

Spontaneous delivery took place on day 22 of pregnancy after which, at 2 days old, litter sizes were standardised for each mother. All maternal measures and pup measurements post-weaning were taken in the fed state at 10 am, with plasma levels being measured from tail blood samples. At 21 days of age, all the pups were weaned onto the 20% (w/w) protein diet until 6 weeks of age when half of the pups were transferred to a high fat diet (Charles River, UK Ltd, Margate, UK, the composition of which % metabolisable energy were 20% from protein, 12% from carbohydrate and 68% from fat, as described (Pearson, S.L. et al., Biochem Biophys Res Commun 229, 752-7. (1996)). Throughout the study all the rats were allowed to eat ad libitum and had free access to drinking water. Further investigations were conducted on male rats that had been fasted overnight prior to commencement of procedures, at age 6 weeks, 6 months and 12 months. The results presented are from the second of two independent experiments which gave similar results.

Glucose Tolerance Test

Intraperitoneal glucose tolerance tests were conducted in rats at 6 weeks, 6 months and 12 months of age. Prior to the procedure rats, were fasted overnight and then dosed with glucose (1 g/kg, i.p). Blood samples were taken from the tail for glucose and insulin measurements at 0, 30, 60, 90, 120 and 180 minutes after glucose injection. Glucose tolerance was assessed in terms of areas under the glucose-time curves.

Plasma Analytes and Pancreatic Hormone Measurements

Fasting plasma insulin and leptin were measured by ELISA (Crystal Chem Inc. immunoassay, Chicago, Ill.). Blood glucose, triglycerides (Sigma-Aldrich Company, Dorset, UK) and NEFA (ASC-ACOD, Wako Chemicals, Neuss, Germany) were measured calorimetrically. Fed plasma corticosterone levels were measured in the dams by enzyme-immunoassay (IDS OCTEIA Corticosterone immunoassay, Immunodiagnostic Systems, Boldon, UK). For determination of pancreatic insulin, pancreas samples were removed as soon as possible after death. After weighing, they were placed into ice cold 180 mMol/l hydrochloric acid in 75% (v/v) ethanol (10 ml/gram) (Eriksson, U. et al., Acta Endocrinol (Copenh) 94, 354-64. (1980)) and minced vigorously. The hormones were extracted overnight at 4° C. and the extract separated from the remaining pancreatic tissue by centrifugation at 1800 g for 20 min. Pancreatic insulin content was measured using a radioimmunoassay with rat insulin standards and an antiserum raised against rat insulin. The inter-assay imprecision was 4.8% and the intra-assay imprecision was 1.8%.

Placental 11β-Hydroxysteroid Dehydrogenase Activity

There are two isozymes of 11β-HSD. Both are expressed in placenta, though only the type 2 isozyme inactivates glucocorticoids. Both were assayed. Placentas were homogenised in ice-cold PBS (pH7.4) containing 0.25M sucrose and assayed for 11β-dehydrogenase activity, as described. After a 10 min incubation, steroids were extracted with ethyl acetate and analysed with thin layer chromatography and high pressure liquid chromatography against known standards (Waddell, B. J. et al., Endocrinology 139, 1517-23. (1998)).

Statistics

Glucose tolerance, plasma levels and pancreatic hormone measurements were analysed using Dunnett's Multiple comparison one-way analysis of variance (ANOVA). Results are presented as means ±s.e.m.

1. A method for the prophylaxis or treatment, of a metabolic disorder or other condition developing later in life and associated with low birth weight, said method comprising administering lettin, or a fragment or mimetic thereof to at least one of
(i) an infant of low birth weight for age;
(ii) a nursing mother of an infant, the infant having low birth weight for age;
or -(iii) a pregnant female predisposed to civing birth to an infant of low birth weight for age.

2. The method of claim 1, wherein the condition is type 2 diabetes, obesity, cardiovascular disease, gestational diabetes, impaired glucose tolerance, insulin resistance, hypertension or syndrome X.

3. The method of claim 1, wherein the leptin, or fragment or mimetic thereof, is provided to the infant in milk.

4. The method of claim 1, wherein the leptin, or fragment or mimetic thereof, is provided to the pregnant female during the third trimester of pregnancy.

5. The method of claim 1, wherein the leptin, or fragment or mimetic thereof, is administered to humans, or domestic animals.

6. The method of claim 5, wherein the said domestic animals are cats or dogs.

7. The method of claim 1, wherein the leptin, or fragment or mimetic thereof, is formulated for administration in conjunction with an agent effective for reducing the cortisol or corticosterone bioactivity.

8. The method of claim 7, wherein the agent is a direct antagonist of cortisol or corticosterone.

9. The method of claim 7 wherein the agent increases the rate of inactivation of cortisol or corticosterone.

10. The method of claim 9, wherein the agent increases the rate of conversion of cortisol to cortisone or corticosterone to dehydrocortocosterone.

11. The method of claim 10, wherein the agent stimulates HSD-2 activity or expression.

12. The method of claim 7, wherein the agent decreases the rate of synthesis of cortisol or corticosterone.

13. The method of claim 12, wherein the agent decreases the rate of conversion of cortisone to cortisol or dehydrocorticosterone to corticosterone.

14. The method of claim 13, wherein the agent inhibits HSD-1 activity or expression.

15. A method for the prophylaxis, of a metabolic disorder or other condition developinq later in life and associated with low birth weight, said method comorisinq administerinq an aaent effective for enhancing endocrenous levels of leptin to at least one of
(i) an infant of low birth weight for acre;
(ii) a nursina mother of an infant, the infant having low birth weight for age,
or -(iii) a pregnant female predisPosed to giving birth to an infant of low birth weiaht for aye.

16. A kit comprising leptin, or a fragment or mimetic thereof, and instructions for administration to a pregnant female, a nursing mother, or an infant.

17. A kit according to claim 16, further comprising an agent effective for reducing cortisol or corticosterone bioactivity.

18. A method of preparing a medicament or foodstuff, comprising the step of admixing leptin or a fragment or mimetic thereof, with milk.

19. A method according to claim 18, wherein the leptin, fragment or mimetic thereof is from a first mammalian species, and the milk is from a different source.

20. A method according to claim 19, wherein the milk is from a second mammalian species.

21. A method according to claim 20, wherein the milk is from cows, goats, or sheep.

22. A method according to claim 18, wherein the leptin is human leptin.

23. A method according to claim 18, wherein the leptin is feline or canine leptin.

24. A method according to claim 18, further comprising admixing an agent effective for reducing cortisol or corticosterone bioactivity with said milk.

25. A medicament or foodstuff, comprising leptin, or a fragment or mimetic thereof, from a first mammalian species, and milk from a different source.

26. A medicament or foodstuff according to claim 25, wherein the milk is derived from a second mammalian species.

27. A medicament or foodstuff according to claim 26, wherein the milk is from cows, goats, or sheep.

28. A medicament or foodstuff according to claim 25, wherein the leptin is human leptin.

29. A medicament or foodstuff according to claim 25, wherein the leptin is feline or canine leptin.

30. A medicament or foodstuff according to claim 25, further comprising an agent effective for reducing cortisol or corticosterone bioactivity.
